(19) United States
(12) Patent Application Publication
(10) Pub. No.: US 2017/0122708 A1
(43) Pub. Date: May 4, 2017

(54) WEARABLE PERSONAL PROTECTION SPRAY DEVICE WITH TRIGGER AND SAFETY MECHANISM

(71) Applicant: Defendables, LLC, Lawrenceville, GA (US)

(72) Inventors: Kristine R. Gorinas, Lawrenceville, GA (US); Eric W. Huber, San Juan Capistrano, CA (US)

(21) Appl. No.: 15/408,955

(22) Filed: Jan. 18, 2017

Related U.S. Application Data
(63) Continuation-in-part of application No. 15/230,328, filed on Aug. 5, 2016.
Application No. 15/230,328 is a continuation-in-part of application No. 15/165,755, filed on May 26, 2016, now abandoned.
(60) Provisional application No. 62/282,664, filed on Aug. 8, 2015.
(60) Provisional application No. 62/285,908, filed on Nov. 12, 2015.
(60) Provisional application No. 62/230,165, filed on May 29, 2015.

(51) Int. Cl. F41H 9/10; B65D 83/20; B65D 83/14; A44C 9/00; A61K 31/165; A61K 9/12; A44C 15/00; A44C 5/00; B65D 83/38; A61K 31/12

(52) U.S. Cl. CPC F41H 9/10; B65D 83/38; B65D 83/20; B65D 83/752; A61K 31/12; A61K 31/165; A61K 9/12; A44C 15/002; A44C 5/0007; A44C 15/005; A44C 9/0061

(57) ABSTRACT

A fluid dispenser apparatus includes a sealed fluid reservoir container storing a volume of a fluid under a pressure higher than at least 140 psi, a nozzle juxtaposed with the sealed fluid reservoir container, and a trigger mechanically coupled with the nozzle. The nozzle has a fluid inlet that is configured to fluidly couple with the volume of the sealed fluid reservoir container and a fluid outlet configured to emit a stream of the fluid. When activated the fluid inlet of the nozzle is caused to permanently unseal the sealed fluid reservoir container and relate substantially all the fluid under pressure in the volume through the fluid outlet as the stream in a direction determined by a user.

### CROSS-REFERENCE TO RELATED APPLICATION

The present application is a U.S. continuation-in-part patent application of, and claims priority under 35 U.S.C. §120 to, U.S. nonprovisional patent application Ser. No. 15/230,328, filed Aug. 5, 2016, which nonprovisional patent application is:
(1) a U.S. continuation-in-part patent application of, and claims priority under 35 U.S.C. §120 to, U.S. nonprovisional patent application Ser. No. 15/165,755, filed May 26, 2016, which nonprovisional patent application is a U.S. nonprovisional patent application of, and claims priority under 35 U.S.C. §119(e) to, U.S. provisional patent application Ser. No. 62/230,165 filed May 29, 2015;
(2) a U.S. nonprovisional patent application of, and claims priority under 35 U.S.C. §119(e) to, U.S. provisional patent application Ser. No. 62/282,664, filed Aug. 8, 2015; and
(3) a U.S. nonprovisional patent application of, and claims priority under 35 U.S.C. §119(e) to, U.S. provisional patent application Ser. No. 62/285,908, filed Nov. 12, 2015.
   Each of the foregoing patent applications is incorporated by reference herein.

### COPYRIGHT STATEMENT

All of the material in this patent document is subject to copyright protection under the copyright laws of United States and other countries. The copyright owner has no objection to the facisimle reproduction by anyone of the patent document or the patent disclosure, as it appears in official governmental records but, otherwise, all other copyright rights whatsover are reserved.

### BACKGROUND OF THE PRESENT INVENTION

Field of the Present Invention

The present invention relates generally to a protection type spray, and in particular, to a wearable personal protection spray device with trigger and safety mechanism.

Background

Personal protection sprays are in wide use, but are not typically provided in a convenient wearable package. A need thus exists for improvements in this area.

### SUMMARY OF THE PRESENT INVENTION

Some exemplary embodiments of the present invention may overcome one or more of the above disadvantages and other disadvantages not described above, but the present invention is not required to overcome any particular disadvantage described above, and some exemplary embodiments of the present invention may not overcome any of the disadvantages described above.

Broadly defined, the present invention according to one aspect is a wristband-mounted wearable personal protection spray unit, including: a wristband; a slide cartridge carried on the wristband; a vessel, containing personal protection spray, that fits inside the slide cartridge; and a mechanism, integrated with the wristband, for activating the vessel to release the personal protection spray contained therein.

In a feature of this aspect, the mechanism includes a vessel tab and a cartridge tab that are activated by squeezing them together. In further features, the personal protection spray includes capsaicin and/or the personal protection spray includes phenacyl chloride.

In another feature of this aspect, the personal protection spray is an aerosol self-defense spray.

Broadly defined, the present invention according to another aspect is a fluid dispenser apparatus including: a sealed fluid reservoir container storing a volume of a fluid under pressure; a nozzle juxtaposed with the sealed fluid reservoir container, and having a fluid inlet that is configured to fluidly couple with the volume of the sealed fluid reservoir container and a fluid outlet configured to emit a stream of the fluid; and a trigger mechanically coupled with the nozzle and configured, upon activation, to cause the fluid inlet of the nozzle to permanently unseal the sealed fluid reservoir container and relate substantially all the fluid under pressure in the volume through the fluid outlet as the stream in a direction determined by a user.

In a feature of this aspect, the pressure is at least 80 psi. In further features, the pressure is at least 100 psi; the pressure is at least 130 psi; and/or the pressure is at least 140 psi.

In another feature of this aspect, the fluid reservoir container comprises a non-cylindrical geometry defining the volume.

In another feature of this aspect, the fluid reservoir container comprises a substantially ovoid geometry defining the volume. In a further feature, the ovoid comprises a maximum dimension of no more than 50 mm.

In another feature of this aspect, the fluid reservoir container comprises at least one depression sized and dimensioned to receive at least a portion of the nozzle.

In another feature of this aspect, the volume is no more than about 15 ml. In further features, the volume is no more than about 10 ml; the volume is no more than about 9 ml; the volume is no more than about 8 ml; the volume is no more than about 7 ml; the volume is no more than about 6 ml; the volume is no more than about 5 ml; and/or the volume is no more than about 4 ml.

In another feature of this aspect, the apparatus further includes a housing that contains at least the reservoir. In further features, the housing includes a cover and a detachable base, where the cover releasably couples to the detachable base and permits replacing of the fluid reservoir container; the apparatus further includes a clip holder coupled to the housing; the apparatus further includes an optional clip configured to couple with the clip holder; the apparatus further includes an appendage strap configured to couple to the housing; the appendage strap is sized and dimensioned strap the housing to a wrist; the appendage strap comprises a hook-and-loop fastener; the fluid inlet includes a needle portion configured to pierce the sealed fluid upon activation by the trigger; and/or the fluid inlet is shaped to depress an internal valve releasing the pressurized contents of reservoir upon activation by the trigger.

In another feature of this aspect, the fluid comprises an irritant. In further features, the irritant comprises a pepper derived irritant; and/or the irritant includes capsaicin.

In another feature of this aspect, the fluid comprises a marking component.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

Further features, embodiments, and advantages of the present invention will become apparent from the following detailed description with reference to the drawings, wherein:

FIG. 1 is a perspective view of a wristband-mounted wearable personal protection spray unit, viewed from a trigger side, in accordance with one or more preferred embodiments of the present invention;

FIG. 2 is a perspective view of the wristband-mounted wearable personal protection spray unit of FIG. 1, viewed from a spray side thereof;

FIG. 3 is an exploded view of the primary components of the wristband-mounted wearable personal protection spray unit of FIG. 1;

FIG. 4 is a perspective view focusing on the spray components;

FIG. 5 is a perspective view of the spray vessel;

FIG. 6 is an illustration of a wristband-mounted wearable personal protection spray unit being worn;

FIG. 7 is an illustration showing activation of a wristband-mounted wearable personal protection spray unit;

FIG. 8 is an illustration demonstrating use of a wristband-mounted wearable personal protection spray unit;

FIG. 9 is a perspective, partially schematic view of a wearable spray canister with trigger and safety mechanism in accordance with one or more preferred embodiments of the present invention;

FIG. 10 is an exploded view of the wearable spray canister with trigger and safety mechanism of FIG. 9;

FIG. 11 is a top view of a trigger mechanism and vessel without cover in stand-by mode;

FIG. 12 is a top view of a trigger mechanism and vessel without cover in activated mode;

FIG. 13 is an exploded view of one or more wearable personal protection spray unit in accordance with one or more preferred embodiments of the present invention;

FIG. 14 is a top perspective view of a plurality of wearable personal protection spray units;

FIG. 15 is a series of illustrations of components of a wearable personal protection spray unit and their assembly and/or operation, all in accordance with one or more preferred embodiments of the present invention;

FIG. 16 is a perspective view of a wearable personal protection spray unit in accordance with one or more preferred embodiments of the present invention;

FIGS. 17A & 17B are fragmentary perspective views of wearable personal protection spray units worn on human wrists;

FIG. 18 is a photographic illustration of a jewelry-style wearable personal protection spray unit, worn on a human wrist, in use, all in accordance with one or more preferred embodiments of the present invention;

FIGS. 19-23 are photographic illustrations of jewelry-style form factors for a wearable personal protection spray unit in accordance with one or more preferred embodiments of the present invention;

FIG. 24 is a series of illustrations of components of a wearable personal protection spray unit and their assembly and/or operation, all in accordance with one or more preferred embodiments of the present invention;

FIG. 25 is a series of illustrations of components of a wearable personal protection spray unit and their assembly and/or operation, all in accordance with one or more preferred embodiments of the present invention;

FIG. 26 is a fragmentary perspective view of a wearable personal protection spray unit worn on a human wrist, all in accordance with one or more preferred embodiments of the present invention;

FIG. 27 is a fragmentary perspective view of a wearable personal protection spray unit worn on a human wrist, all in accordance with one or more preferred embodiments of the present invention;

FIG. 28 is a perspective view of another wearable personal protection spray unit in accordance with one or more preferred embodiments of the present invention;

FIGS. 29 and 30 are a side view and a side cross-sectional view, respectively, of the housing and trigger of the wearable personal protection spray unit of FIG. 28;

FIG. 31 is an isometric view of the lower half of the housing of FIG. 28;

FIG. 32 is a front isometric view of the valve assembly of FIG. 28, shown in an operational state;

FIG. 33 is a rear isometric view of the valve assembly of FIG. 28;

FIG. 34 is a side view of the valve assembly of FIGS. 32 and 33;

FIG. 35 is a side cross-sectional view of the valve assembly of FIGS. 32 and 33; and

FIG. 36 is an exploded view of the valve assembly of FIGS. 32 and 33.

### DETAILED DESCRIPTION

As a preliminary matter, it will readily be understood by one having ordinary skill in the relevant art (“Ordinary Artisan”) that the present invention has broad utility and application. Furthermore, any embodiment discussed and identified as being “preferred” is considered to be part of a best mode contemplated for carrying out the present invention. Other embodiments also may be discussed for additional illustrative purposes in providing a full and enabling disclosure of the present invention. As should be understood, any embodiment may incorporate only one or a plurality of the above-disclosed aspects of the invention and may further incorporate only one or a plurality of the above-disclosed features. Moreover, many embodiments, such as adaptations, variations, modifications, and equivalent arrangements, will be implicitly disclosed by the embodiments described herein and fall within the scope of the present invention.

Accordingly, while the present invention is described herein in detail in relation to one or more embodiments, it is to be understood that this disclosure is illustrative and exemplary of the present invention, and is made merely for the purposes of providing a full and enabling disclosure of the present invention. The detailed disclosure herein of one or more embodiments is not intended, nor is to be construed, to limit the scope of patent protection afforded the present invention, which scope is to be defined by the claims and the equivalents thereof. It is not intended that the scope of patent protection afforded the present invention be defined by reading into any claim a limitation found herein that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present invention. Accordingly, it is intended that the scope of patent protection afforded the present invention is to be defined by the appended claims rather than the description set forth herein.

Additionally, it is important to note that each term used herein refers to that which the Ordinary Artisan would understand such term to mean based on the contextual use of such term herein. To the extent that the meaning of a term used herein—as understood by the Ordinary Artisan based on the contextual use of such term—differs in any way from any particular dictionary definition of such term, it is intended that the meaning of the term as understood by the Ordinary Artisan should prevail.

Regarding applicability of 35 U.S.C. §112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. Thus, reference to “a picnic basket having an apple” describes “a picnic basket having at least one apple” as well as “a picnic basket having apples.” In contrast, reference to “a picnic basket having a single apple” describes “a picnic basket having only one apple.”

When used herein to join a list of items, “or” denotes “at least one of the items,” but does not exclude a plurality of items of the list. Thus, reference to “a picnic basket having cheese or crackers” describes “a picnic basket having cheese without crackers,” “a picnic basket having crackers without cheese,” and “a picnic basket having both cheese and crackers.” Finally, when used herein to join a list of items, “and” denotes “all of the items of the list.” Thus, reference to “a picnic basket having cheese and crackers” describes “a picnic basket having cheese, wherein the picnic basket further has crackers,” as well as describes “a picnic basket having crackers, wherein the picnic basket further has cheese.”

Referring now to the drawings, in which like numerals represent like components throughout the several views, one or more preferred embodiments of the present invention are next described. The following description of one or more preferred embodiments is merely exemplary in nature and is in no way intended to limit the invention, its implementations, or uses.

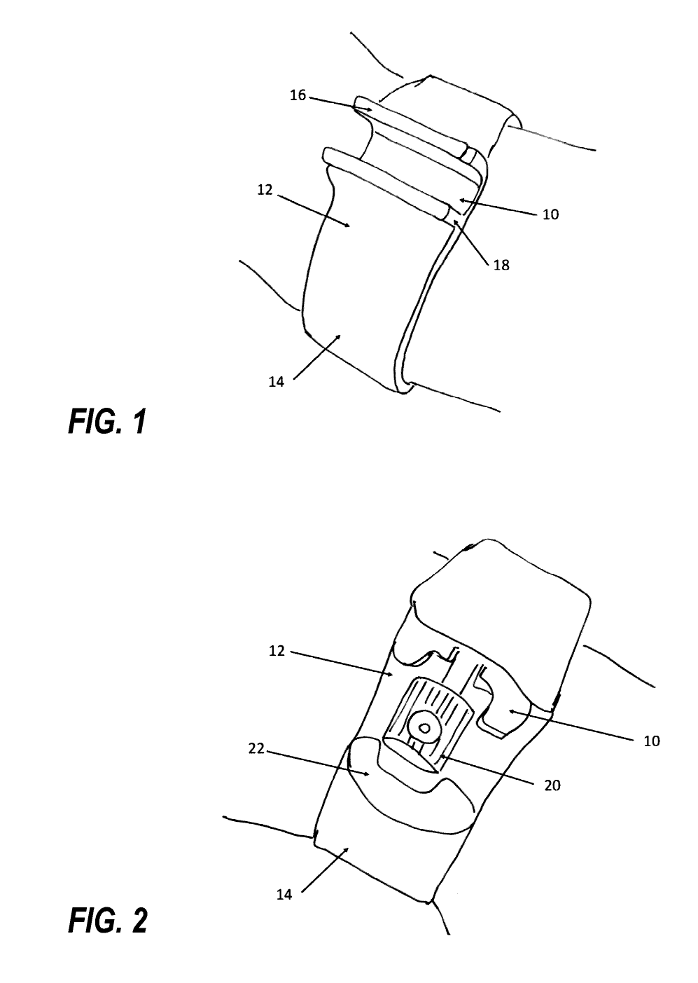

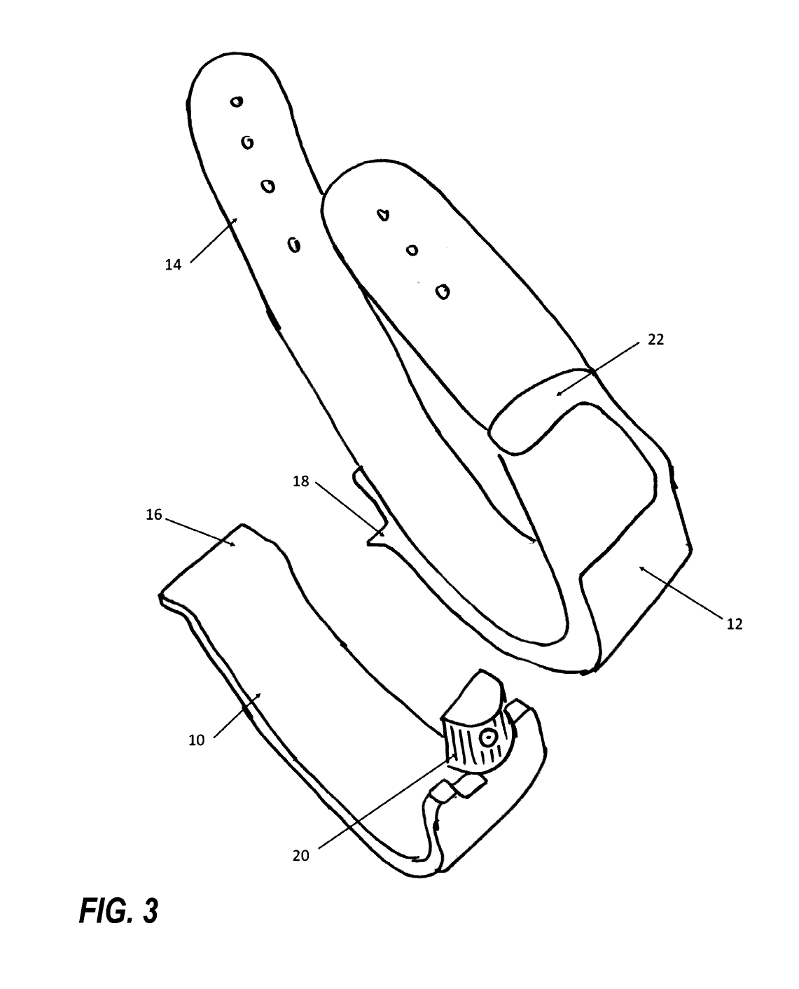

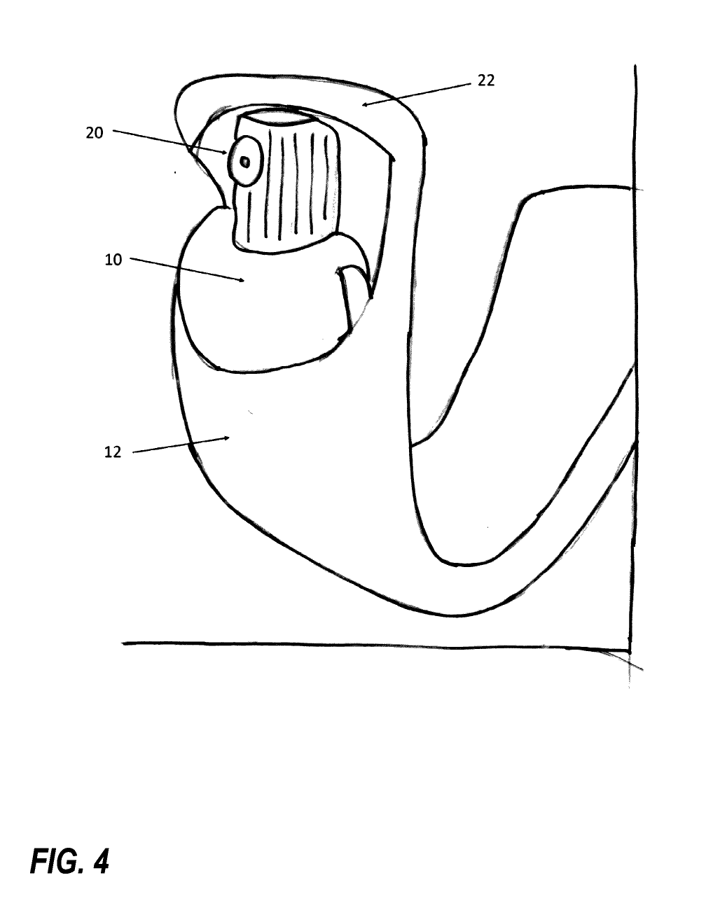

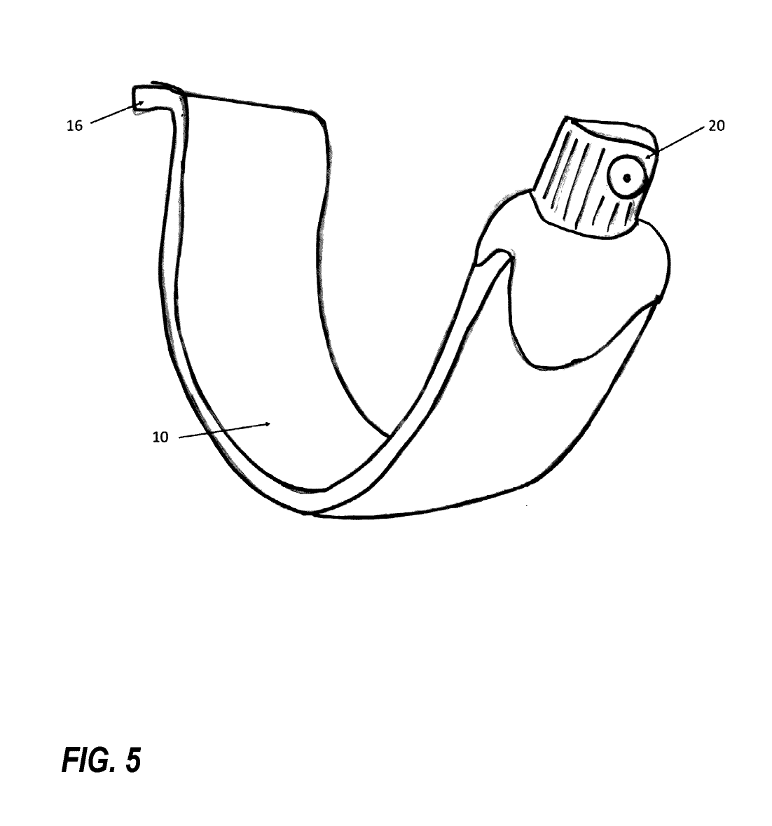

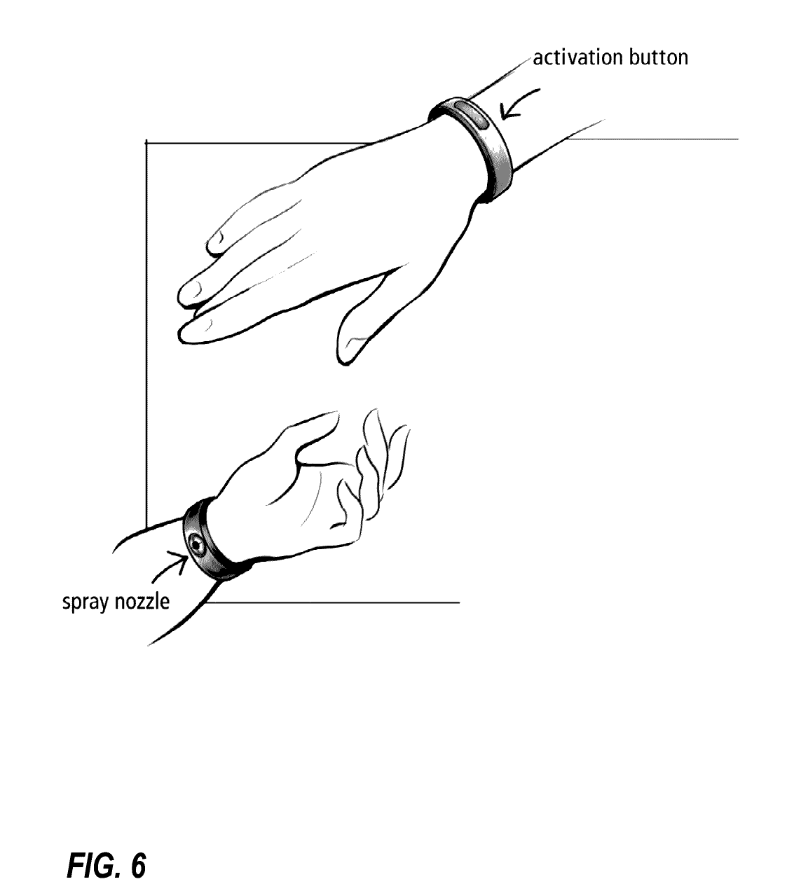

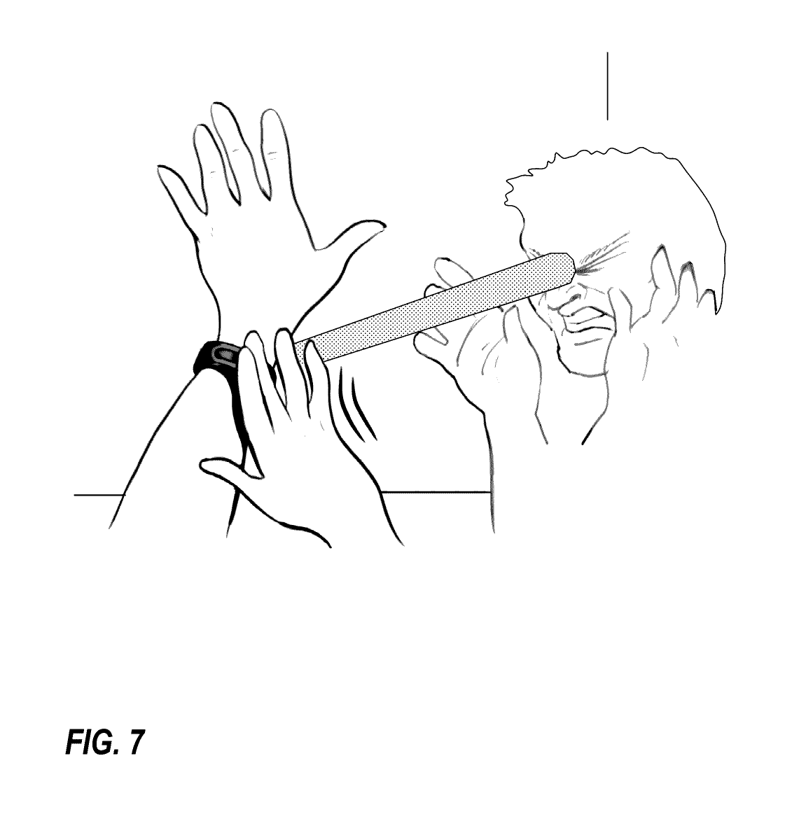

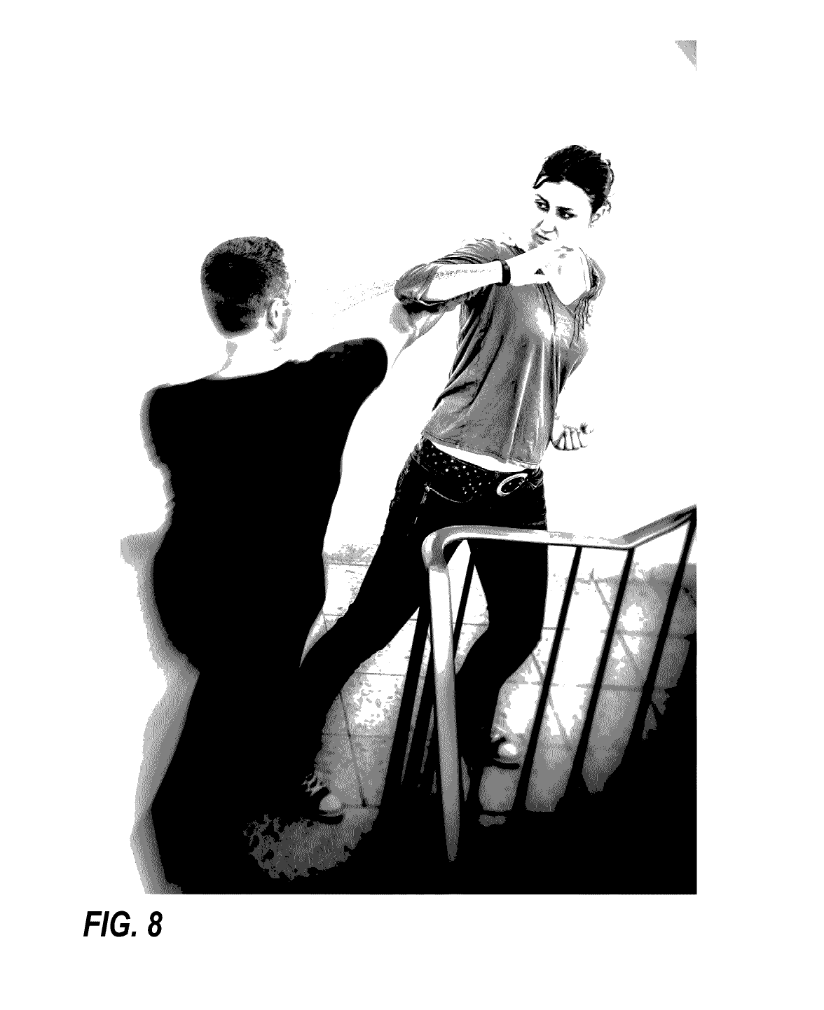

FIG. 1 is a perspective view of a wristband-mounted wearable personal protection spray unit, viewed from a trigger side, in accordance with one or more preferred embodiments of the present invention. FIG. 2 is a perspective view of the wristband-mounted wearable personal protection spray unit of FIG. 1, viewed from a spray side thereof. FIG. 3 is an exploded view of the primary components of the wristband-mounted wearable personal protection spray unit of FIG. 1. FIG. 4 is a perspective view focusing on the spray components. FIG. 5 is a perspective view of the spray vessel. FIG. 6 is an illustration of a wristband-mounted wearable personal protection spray unit being worn. FIG. 7 is an illustration showing activation of a wristband-mounted wearable personal protection spray unit. FIG. 8 is an illustration demonstrating use of a wristband-mounted wearable personal protection spray unit.

The contents vessel 10 fits inside the slide cartridge 12 and freely moves longitudinally to each other. The slide cartridge 12 is integrated to the wrist band 14, shown in more detail in FIG. 3.

At one end of the contents vessel 10, shown in more detail in FIG. 5, positioned at an approximate right angle to its body is the vessel tab 16, shown in more detail in FIG. 1. At the other end of the contents vessel 10 is the spray nozzle 20, shown in more detail in FIG. 2.

At one end of the slide cartridge 12, positioned at an approximate right angle to its body is the cartridge tab 18, shown in more detail in FIG. 1. At the other end of the slide cartridge 12 is the nozzle stop 22, shown in more detail in FIG. 2.

For personal self-protection, the user attaches the invention onto their wrist using the wrist band 14. It is orientated with spray nozzle on the bottom side of user's wrist and the vessel tab 16 and cartridge tab 18 at the top side of the wrist.

When the user squeezes the vessel tab 16 and the cartridge tab 18 together the contents vessel 10 slides within slide cartridge 12 causing the spray nozzle 20 to press against the nozzle stop 22, releasing the pepper spray or similar contents that is contained in the contents vessel 10.

In at least some embodiments, but not necessarily all, instead of a mechanical means, an electronic ignition can be used to start the self-defense spray.

In at least some embodiments, but not necessarily all, other shapes are used for the vessel for the chemical and propellant. These could include a round, tube-like design that might use multiple, side-by-side tubes connected.

In at least some embodiments, but not necessarily all, a valve is used that punctures the vessel, releasing its contents.

In at least some embodiments, but not necessarily all, instead of being worn on the wrist, the invention can be worn in places including, but not limited to around the neck, attached to top, on belt, on hat or as part of other products like water bottles, and purse.

In at least some embodiments, but not necessarily all, the vessel can be designed to be refillable or prefilled vessels can be purchased to replace spent one.

In at least some embodiments, but not necessarily all, electronics can be integrated into the invention, providing common features currently found on existing wearables.

In at least some embodiments, but not necessarily all, a feature that contacts the 911 emergency network, sending GPS coordinates when self-defense feature is activated.

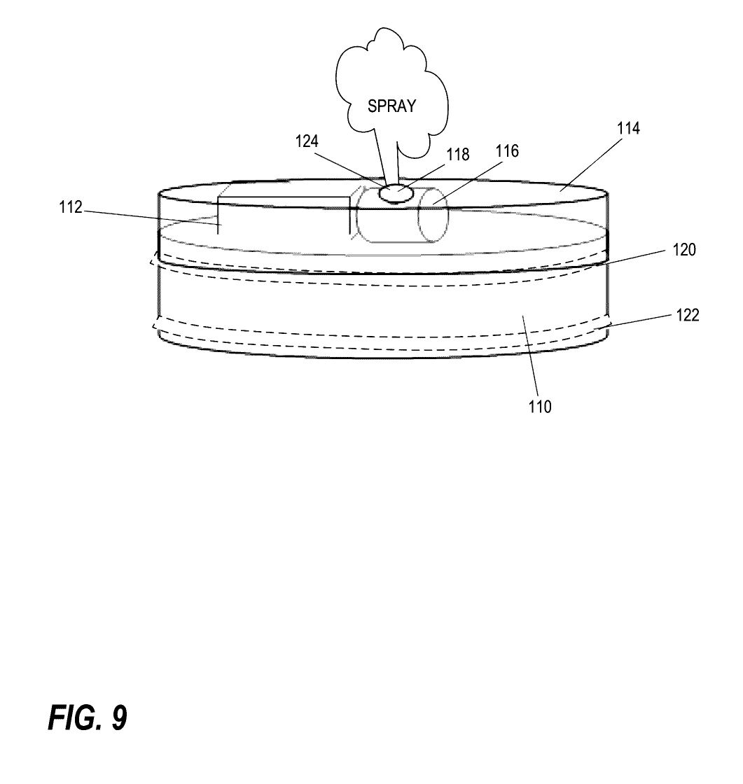

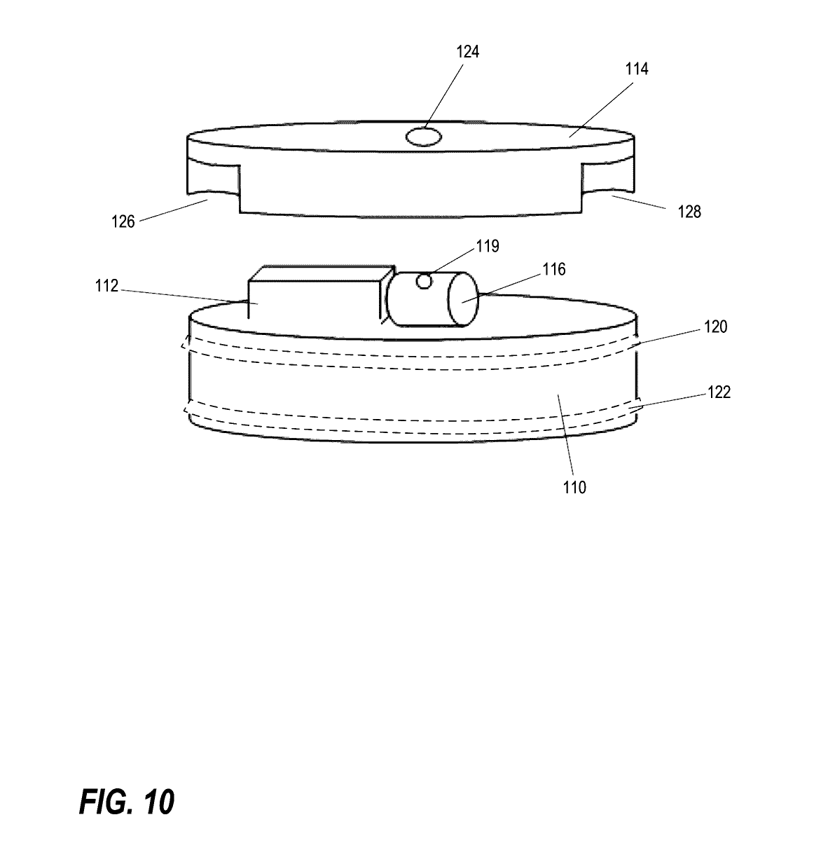

FIG. 9 is a perspective, partially schematic view of a wearable spray canister with trigger and safety mechanism in accordance with one or more preferred embodiments of the present invention, and FIG. 10 is an exploded view of the wearable spray canister with trigger and safety mechanism of FIG. 9.

Positioned on the exterior, a top vessel 110, is the valve housing 112, which together form a single interior cavity. Inserted into the valve mechanism contained in the valve housing 112 is an actuator 116 that when pressed allows the release of the contents trough the spray insert 118. This is shown in some detail in FIGS. 9 and 10.

In FIG. 9 a cover 114 is shown connected to the vessel 110 via a cover connection groove 120. Positioned on the plane surface is the spray opening 124. The spray opening 124 is located at the spot on the cover 114 directly a top the position of the spray insert 118 when the actuator 116 is triggered. Adjacent to the cover connection groove 120 is a housing connection groove 122, which may be a means to add a peripheral like a clip, wrist band, necklace, adhesive or other housing for functional and/or aesthetic purposes. Also, shown on cover 114 is a safety tab opening 126 and a trigger tab opening 128 which, when cover 114 is seated, allows a trigger tab 130 and a safety tab 132 to partially protrude beyond the circumference defined by the vessel 110. This embodiment describes an indentation around the exterior of vessel 110, but other attachment methods are possible.

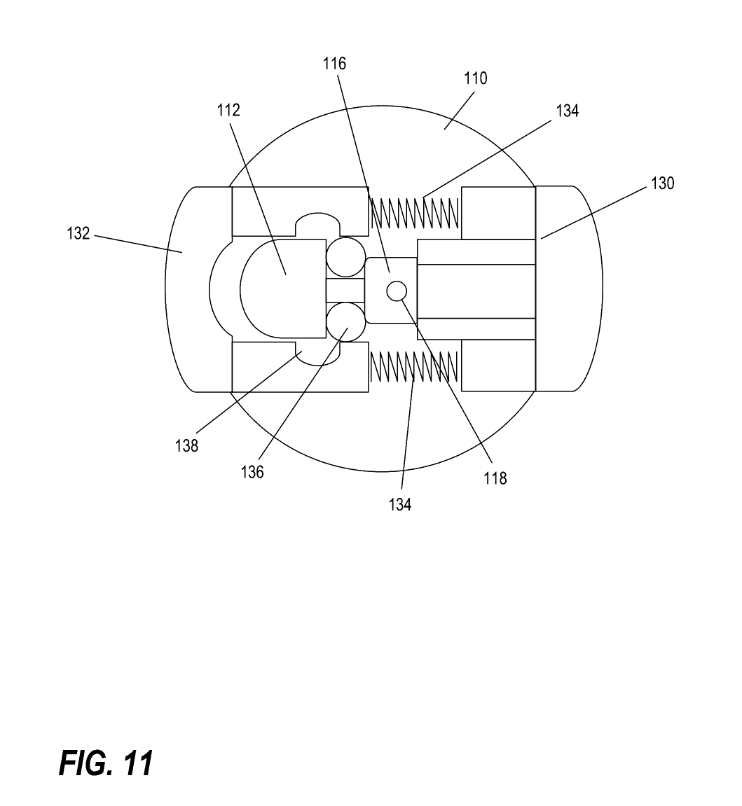

Attached to actuator 116 is the trigger tab 130 which slides parallel to the top plane of the vessel 110 toward the valve housing 112. Similarly the safety tab 132, also moves along the top plane of vessel 110 towards the opposite side of the valve housing. Positioned on the safety tab 132 are indentations identified as obstruction relief 138. Positioned adjacent and independent moving is a safety obstruction 136. Spring 134 are secured in the longitudinal centers providing resistance at one end against the trigger tab 130 and at the other end the safety tab 132. FIG. 11 shows the assembly in its stand-by mode at the ready with the springs 134 relaxed.

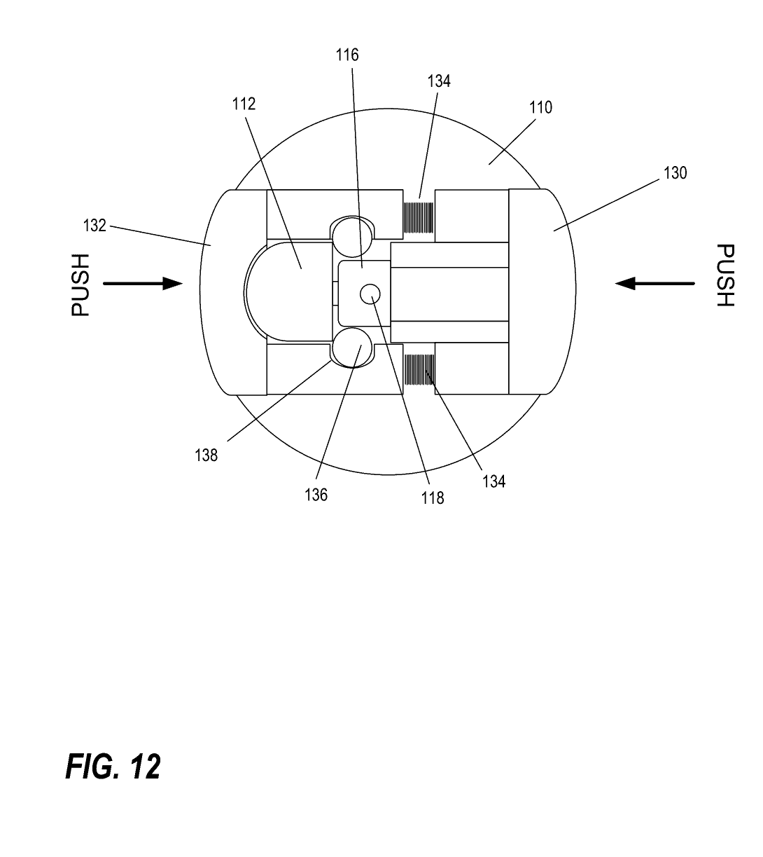

FIG. 12 illustrates the assembly in its active mode with the springs 134 compressed and the safety obstruction 136 seated in the obstruction relief 138 allowing the actuator 116 to be pressed towards the valve housing 112.

To activate this invention for personal protection, the user squeezes the trigger tab 130 and safety tab 132 towards each other. The spring 134 keeps the tabs in stand-by mode until activated by the user.

To prevent accidental activation, the trigger tab 130 is blocked from movement by the safety obstruction 136. Movement is preferably only permitted when the trigger tab 130 and the safety tab 132 are pressed simultaneously. When this happens the safety tab 132 moves the obstruction relief 138 cutout directly adjacent to the safety obstruction 136. The movement of the trigger tab 130 towards the valve housing 112 forces the actuator 116 to pushes away the safety obstruction 136 into the obstruction relief 138 allowing the actuator 116 to open the value in the valve housing 112, releasing the contents, which is under pressure, from the interior of the vessel 110 through the spray insert 118. Based on the content's properties, the spray insert 118 is designed to maximize the shape and characteristics of the spray.

Contents may include, but are not limited to, one or more chemical irritants such as pepper spray, mace, tear gas, chloroacetophenone, CNB, CR, red gel, and criminal identifying gel.

In at least some embodiments, but not necessarily all, instead of a mechanical means, an electronic ignition can be used to start the self-defense spray.

In at least some embodiments, but not necessarily all, a trigger mechanism is included that operates by the pulling of a cord-like component using fingers or teeth.

In at least some embodiments, but not necessarily all, other shapes are used for the vessel for the chemical and propellant. These could include a round, tube-like design that might use multiple, side-by-side tubes connected.

In at least some embodiments, but not necessarily all, a valve is used that punctures the vessel, releasing its contents.

In at least some embodiments, but not necessarily all, the vessel is designed so it can be refillable.

In at least some embodiments, but not necessarily all, electronics are integrated into the invention, providing common features currently found on existing wearable sensor products.

In at least some embodiments, but not necessarily all, a feature is added that contacts to the 911 emergency network, sending GPS coordinates when self-defense feature is activated.

In at least some embodiments, but not necessarily all, a loud, audible alarm feature is added that can be activated in conjunction with the invention's primary use or as a separate feature.

In at least some embodiments, but not necessarily all, a feature is included that allows the invention to be electronically tethered to a smart phone.

In at least some embodiments, but not necessarily all, an advantage is the non-cylindrical shape, diminutive size allows it to be discreetly worn by the user.

In at least some embodiments, but not necessarily all, an advantage is how the invention's trigger mechanism enables easy, controlled use, but with a safety feature that prevents accidental discharge.

In at least some embodiments, but not necessarily all, an advantage is that the invention can be used in conjunction with a variety of housings, including, but not limited to wrist band, bracelet, necklace, finger ring, adhesive-backed and clip.

In at least some embodiments, but not necessarily all, an advantage is the cover can be in different colors, shapes, designs, and materials.

In at least some embodiments, but not necessarily all, an advantage is that the releasing valve is of a commonly used design. This prevents time-consuming and expensive testing that would be typical with a new, unproven design.

In at least some embodiments, but not necessarily all, an advantage is that the vessel, trigger mechanism, cover and housings are separate and interchangeable.

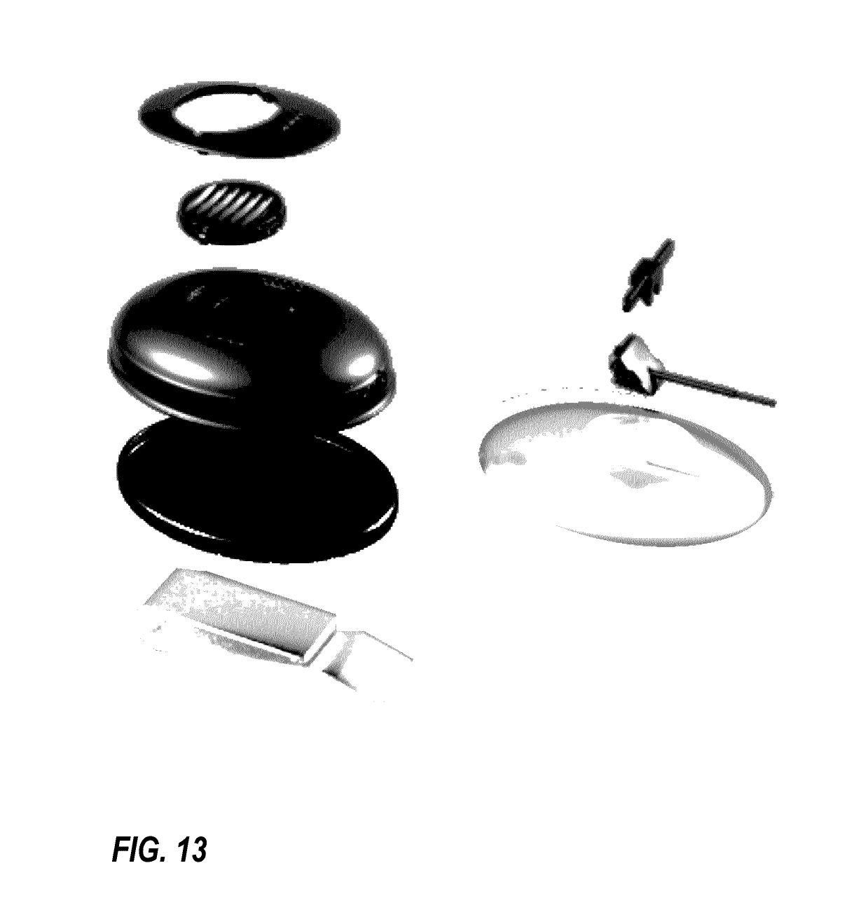

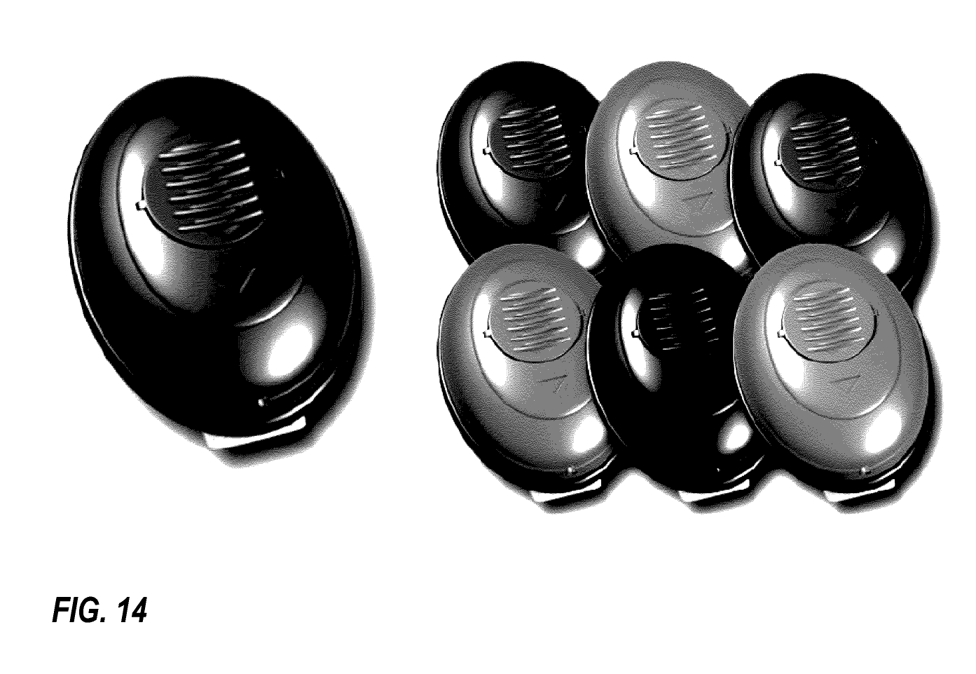

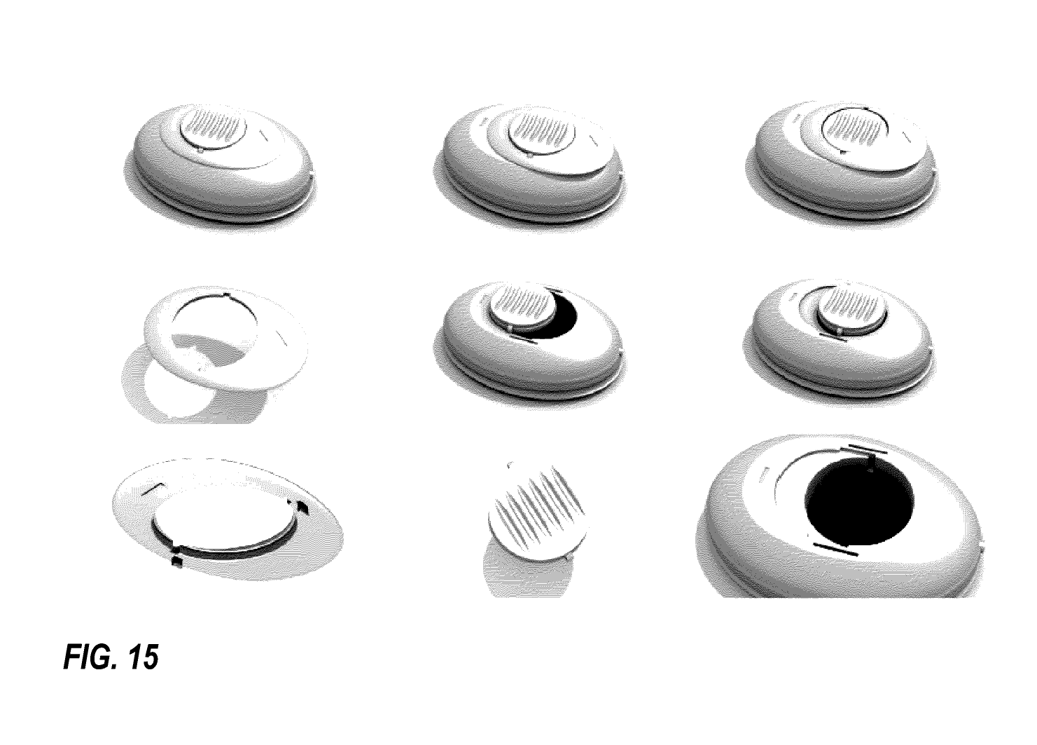

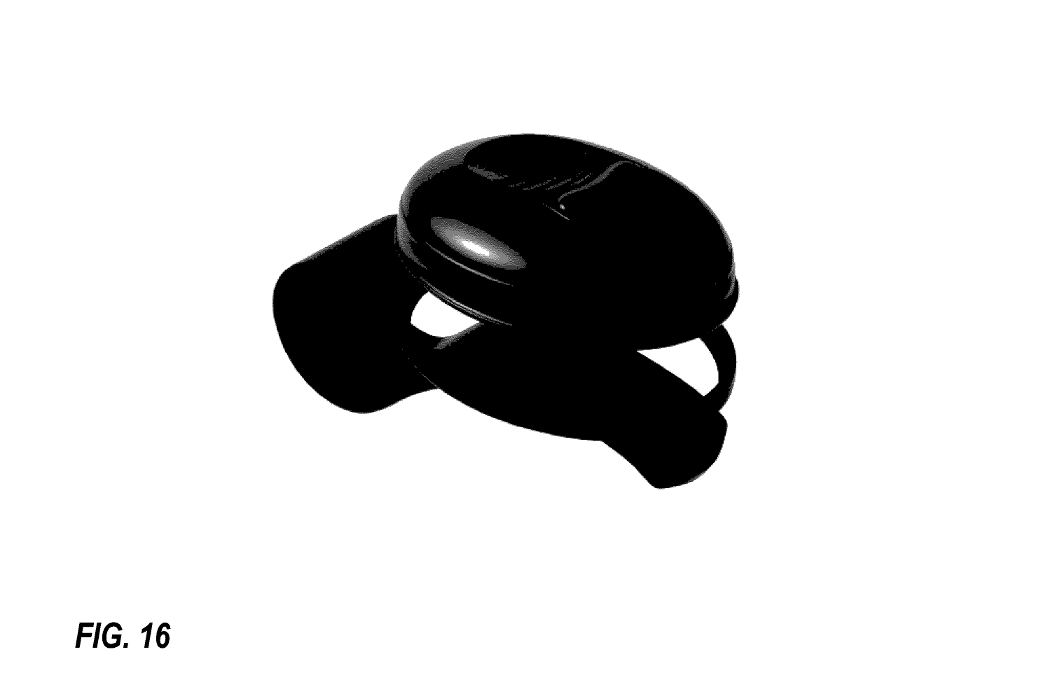

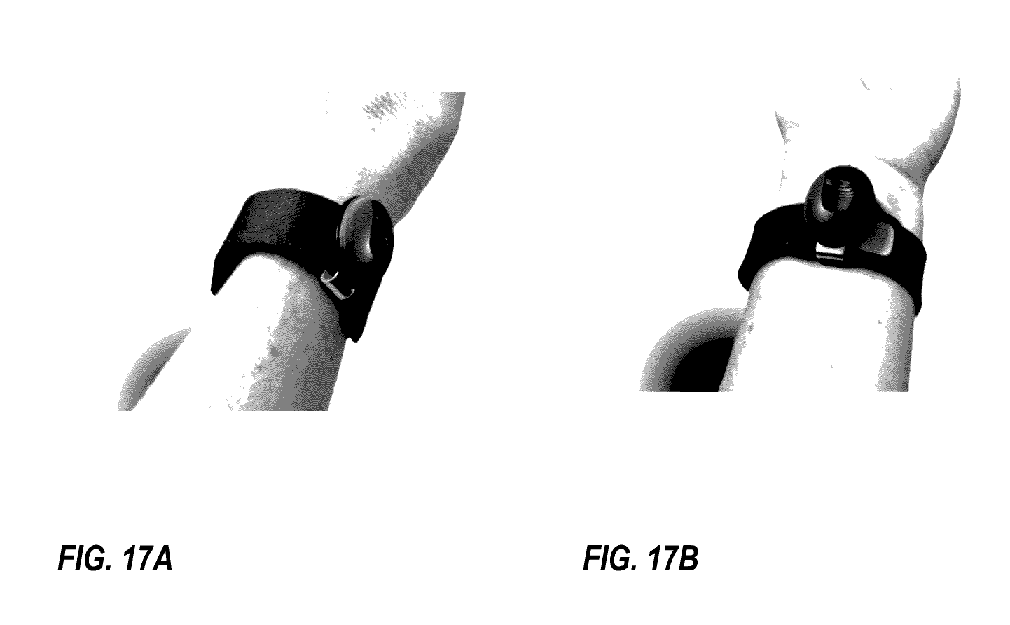

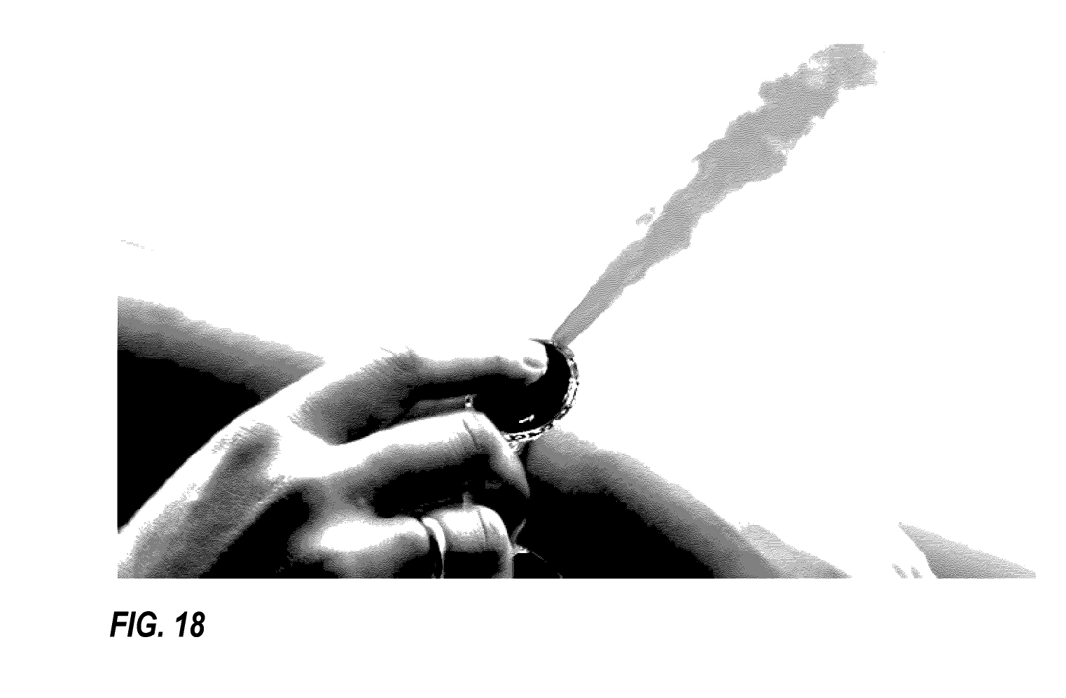

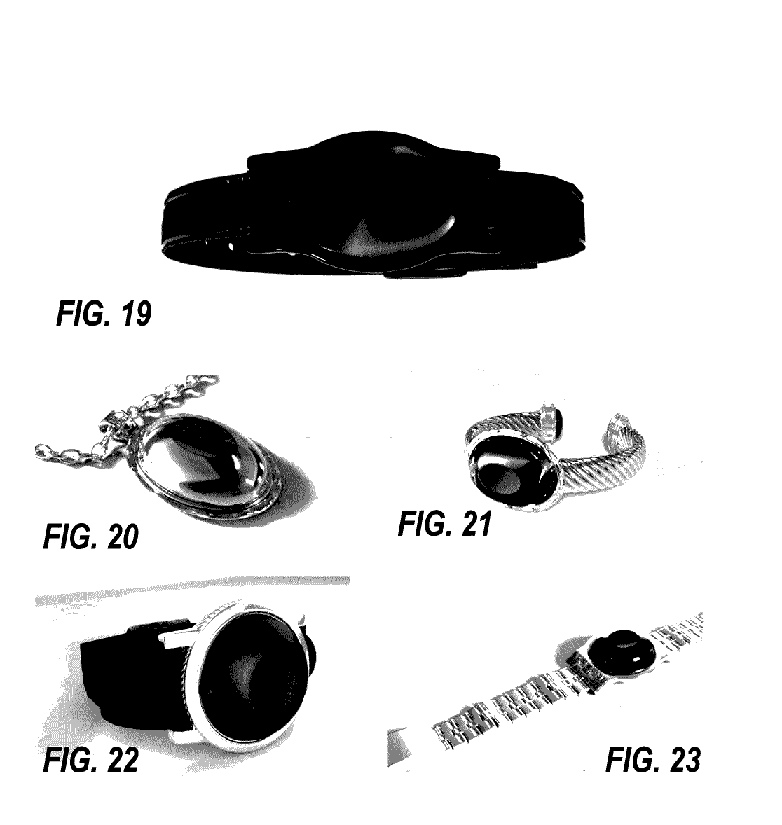

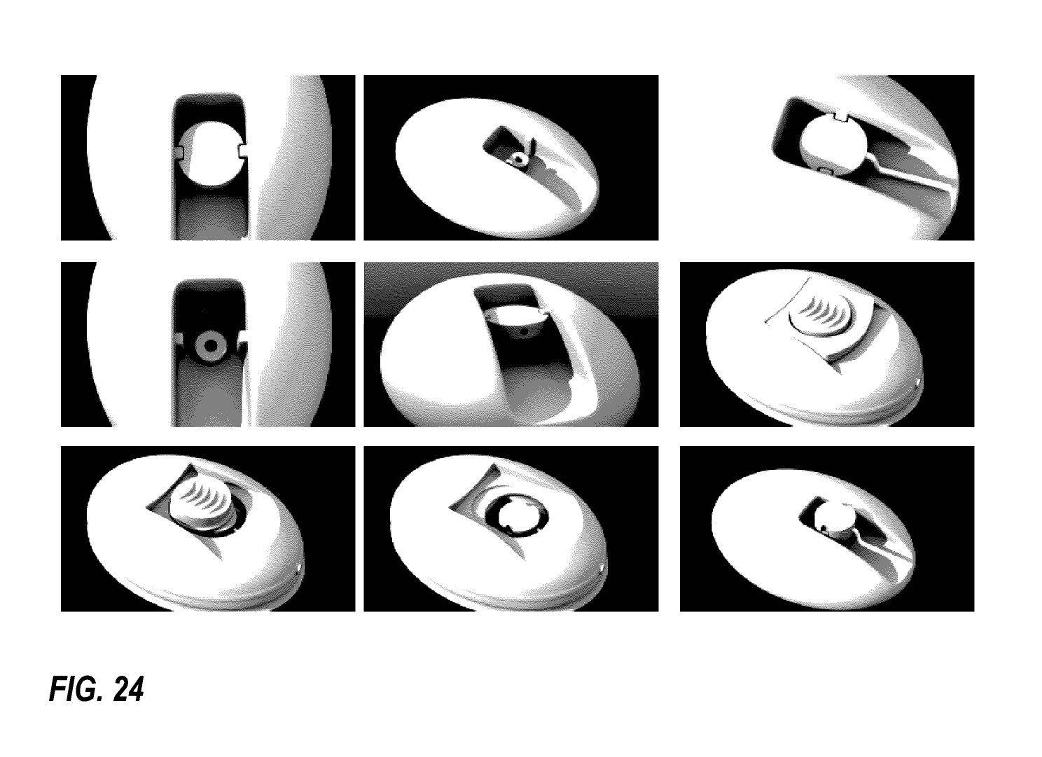

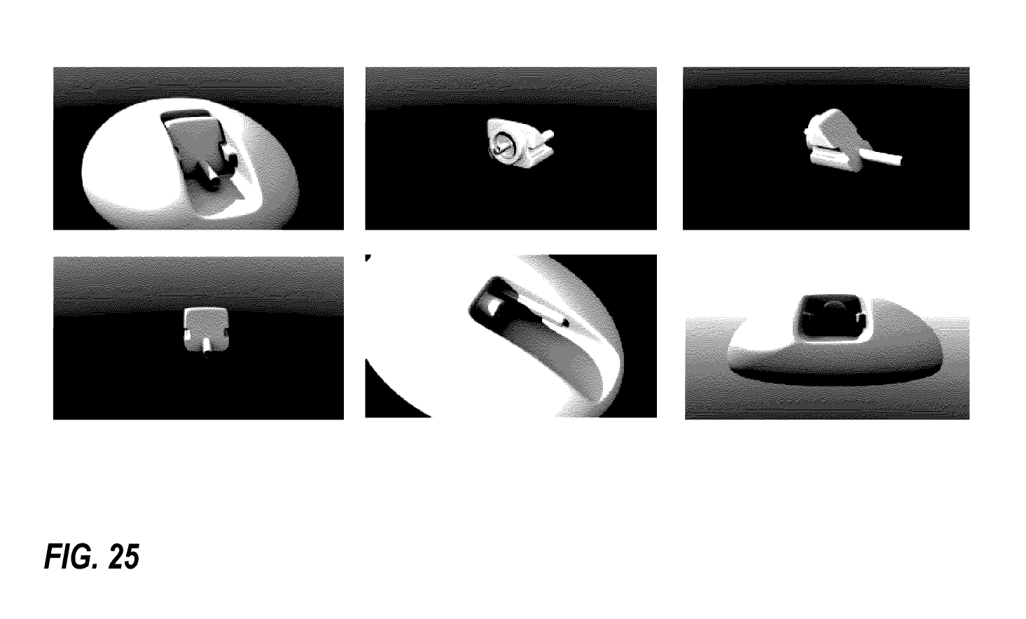

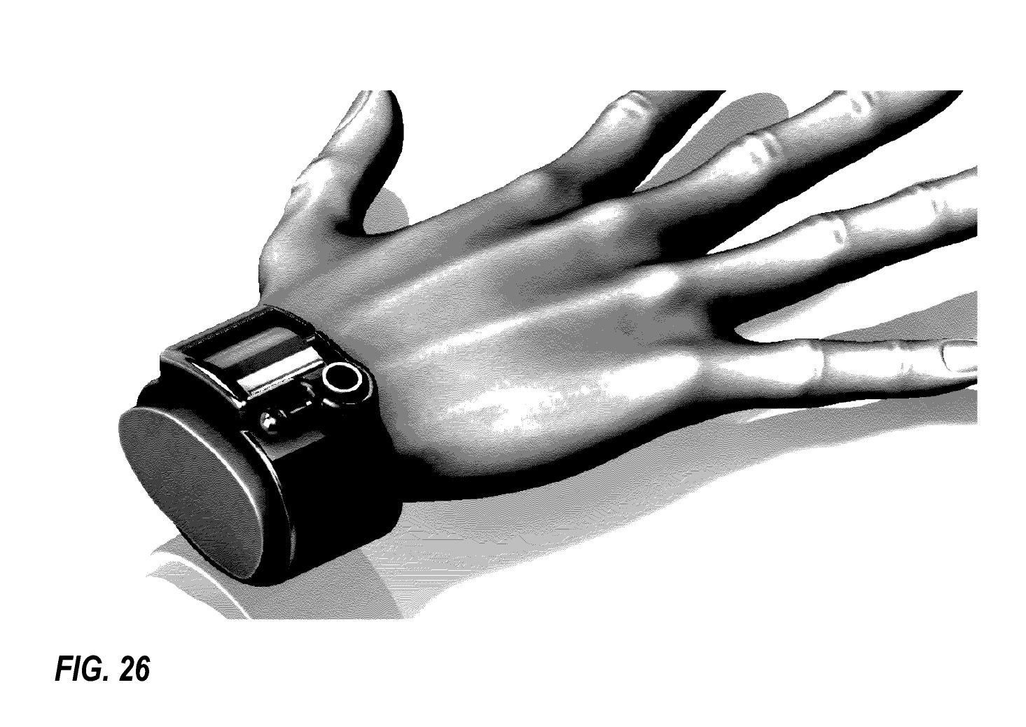

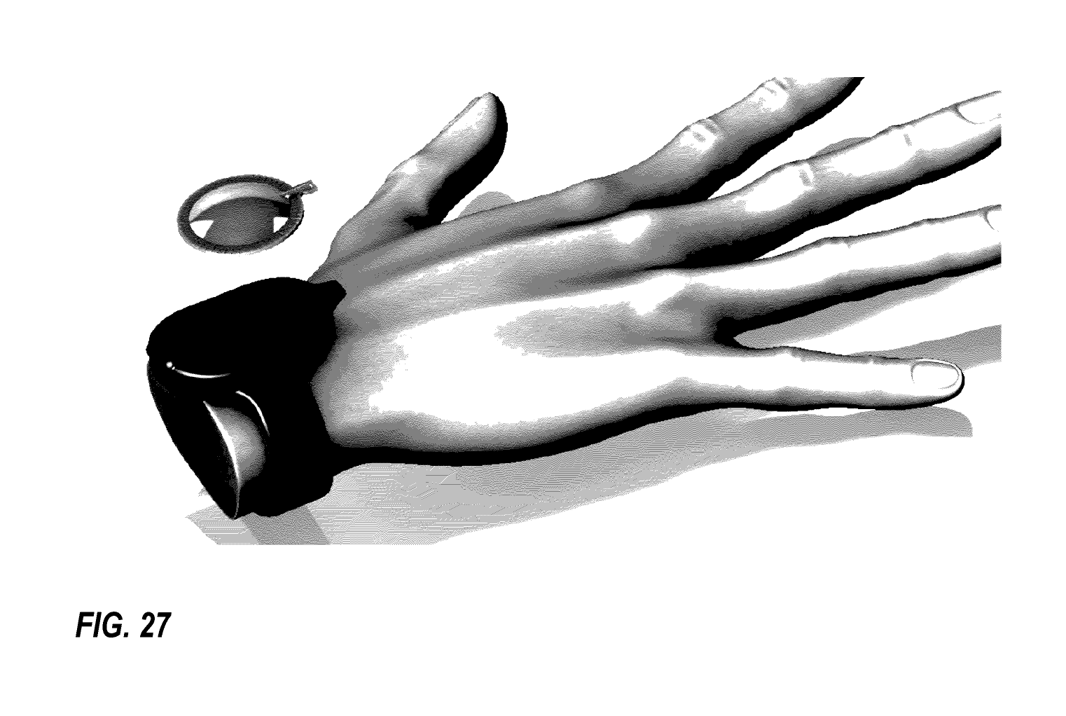

Various designs are possible for a wearable personal protection spray unit. In this regard, FIG. 13 is an exploded view of one or more wearable personal protection spray unit in accordance with one or more preferred embodiments of the present invention; FIG. 14 is a top perspective view of a plurality of wearable personal protection spray units; FIG. 15 is a series of illustrations of components of a wearable personal protection spray unit and their assembly and/or operation, all in accordance with one or more preferred embodiments of the present invention; FIG. 16 is a perspective view of a wearable personal protection spray unit in accordance with one or more preferred embodiments of the present invention; FIGS. 17A & 17B are fragmentary perspective views of wearable personal protection spray units worn on human wrists; FIG. 18 is a photographic illustration of a jewelry-style wearable personal protection spray unit, worn on a human wrist, in use, all in accordance with one or more preferred embodiments of the present invention; FIGS. 19-23 are photographic illustrations of jewelry-style form factors for a wearable personal protection spray unit in accordance with one or more preferred embodiments of the present invention; FIG. 24 is a series of illustrations of components of a wearable personal protection spray unit and their assembly and/or operation, all in accordance with one or more preferred embodiments of the present invention; FIG. 25 is a series of illustrations of components of a wearable personal protection spray unit and their assembly and/or operation, all in accordance with one or more preferred embodiments of the present invention; FIG. 26 is a fragmentary perspective view of a wearable personal protection spray unit worn on a human wrist, all in accordance with one or more preferred embodiments of the present invention; and FIG. 27 is a fragmentary perspective view of a wearable personal protection spray unit worn on a human wrist, all in accordance with one or more preferred embodiments of the present invention.

In various embodiments, a wearable personal protection spray unit is a fluid dispenser apparatus that includes: a sealed fluid reservoir container storing a volume of a fluid under pressure; a nozzle juxtaposed with the sealed fluid reservoir container, and having a fluid inlet that is configured to fluidly couple with the volume of the sealed fluid reservoir container and a fluid outlet configured to emit a stream of the fluid; and a trigger mechanically coupled with the nozzle and configured, upon activation, to cause the fluid inlet of the nozzle to permanently unseal the sealed fluid reservoir container and relate substantially all the fluid under pressure in the volume through the fluid outlet as the stream in a direction determined by a user.

The pressure is preferably at least 80 psi, more preferably at least 100 psi, still more preferably is at least 130 psi, and still more preferably is at least 140 psi.

The fluid reservoir container may include a non-cylindrical geometry defining the volume.

The fluid reservoir container may include a substantially ovoid geometry defining the volume. In at least some embodiments, the ovoid has a maximum dimension of no more than 50 mm.

The fluid reservoir container may include at least one depression that is sized and dimensioned to receive at least a portion of the nozzle.

The volume of the fluid reservoir container is preferably no more than about 15 ml, more preferably no more than about 10 ml, still more preferably no more than about 9 ml, still more preferably no more than about 8 ml, still more preferably no more than about 7 ml, still more preferably no more than about 6 ml, still more preferably no more than about 5 ml, and still more preferably no more than about 4 ml.

In at least some embodiments, the apparatus includes a housing that contains at least the reservoir. The housing may include a cover and a detachable base, where the cover releasably couples to the detachable base and permits replacing of the fluid reservoir container. The apparatus may include a clip holder coupled to the housing. The apparatus may include an optional clip configured to couple with the clip holder. The apparatus may include an appendage strap configured to couple to the housing; such an appendage strap may be sized and dimensioned to strap the housing to a wrist, and may comprise a hook-and-loop fastener.

The fluid inlet may include a needle portion configured to pierce the sealed fluid upon activation by the trigger. The fluid inlet may be shaped to depress an internal valve releasing the pressurized contents of reservoir upon activation by the trigger.

In another feature of this aspect, the fluid comprises an irritant. In further features, the irritant comprises a pepper derived irritant; and/or the irritant includes capsaicin.

In another feature of this aspect, the fluid comprises a marking component.

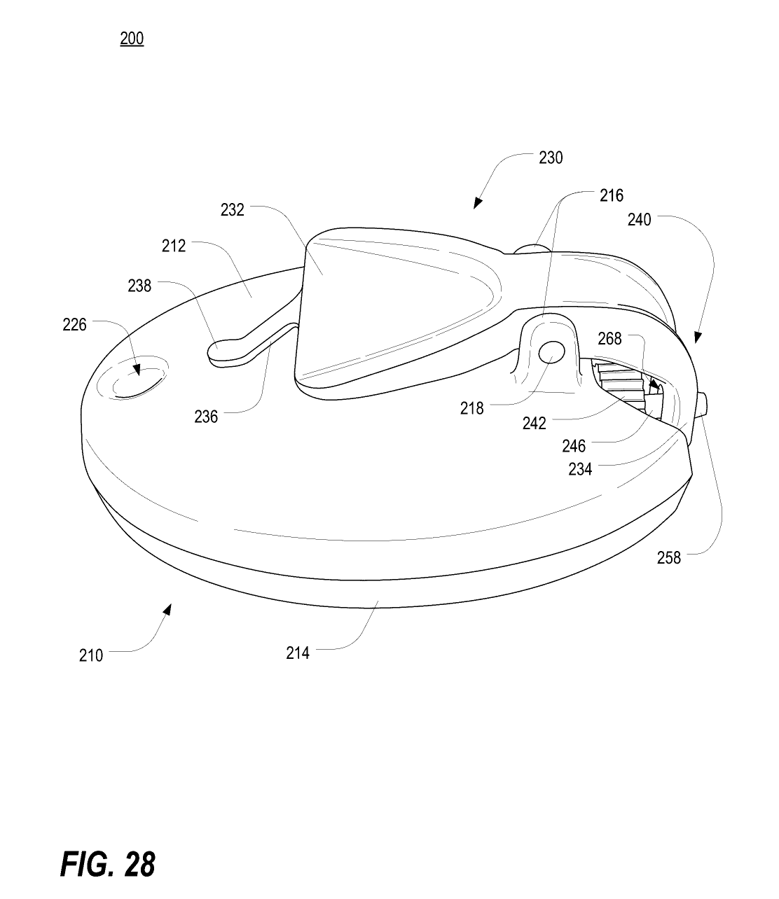

FIG. 28 is a perspective view of another wearable personal protection spray unit 200 in accordance with one or more preferred embodiments of the present invention. As shown therein, the unit 200 includes a housing 210, a trigger 230, and a valve assembly 240.

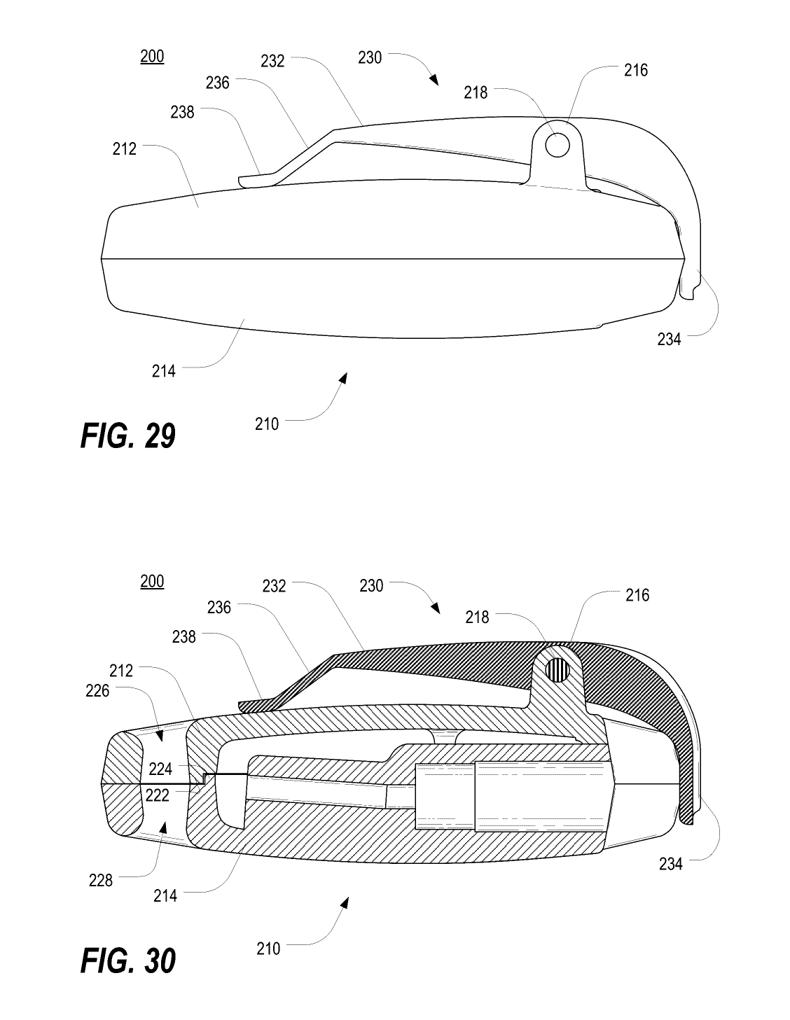

FIGS. 29 and 30 are a side view and a side cross-sectional view, respectively, of the housing 210 and trigger 230 of the wearable personal protection spray unit 200 of FIG. 28. As shown therein, the housing 210 includes a top half 212 and a bottom half 214. Extending upward from the exterior of the top half 212 are a pair of ears 216 supporting an axle 218 on which the trigger 230 is mounted. The interior of each of the top half 212 and the bottom half 214 is generally hollow. Contained within and supported by one or both of the top and bottom halves 212,214 are structures defining a series of cylindrical chambers. In the illustrated embodiment, the cylindrical chambers are molded into the bottom half 214, but it will be appreciated that other arrangements are likewise possible without departing from the scope of the invention. Furthermore, in some embodiments, one or more of the chambers are not cylindrical. The chambers are provided to support portions of the valve assembly 240 and to provide fluid communication between and/or among the interior of the housing 210, the valve assembly 240, and the environment.

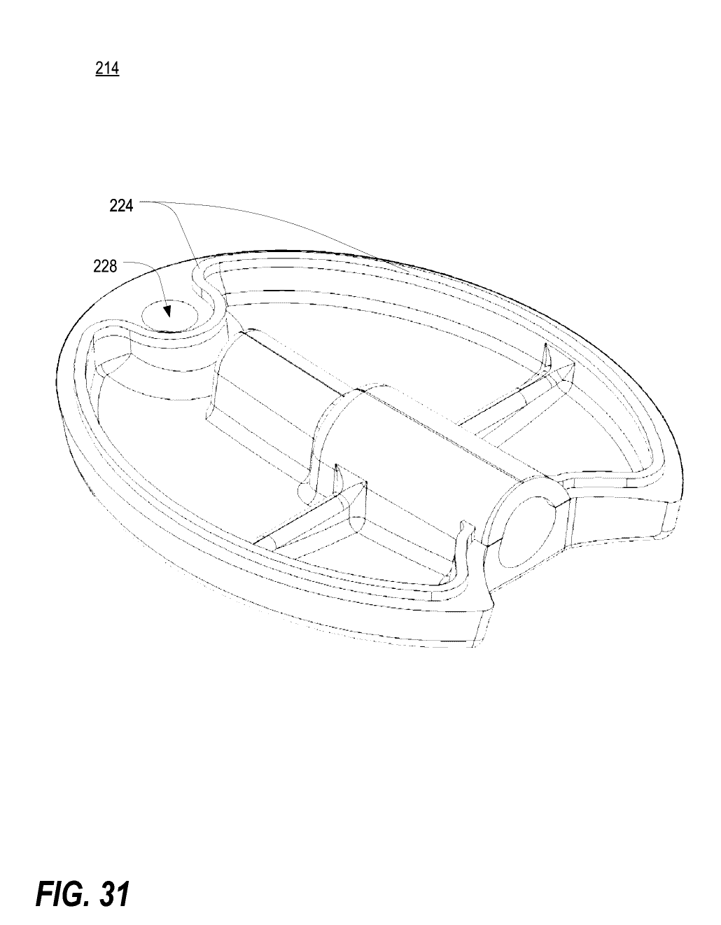

FIG. 31 is an isometric view of the lower half of the housing of FIG. 28. As perhaps best shown in FIGS. 30 and 31, the top half 212 couples to the bottom half 214 via interlocking ridges 222,224. In at least some embodiments the top and bottom halves 212,214 are sealed to one another such that a personal protection fluid may be stored in the hollow interior of the housing 210 without spilling, leaking, or the like.

As perhaps best shown in FIG. 30, a pair of corresponding openings 226,228 penetrate the top half 212 and the bottom half 214 such that an opening extends all the way through the housing 210. This permits the unit 200 to be carried on a chain, clip, or the like.

The trigger 230 is rotatably connected to the axle 218 between the two ears 216 extending from the top of the top housing half 212. The trigger 230 includes a finger actuator 232 and a forked valve interface 234 disposed at opposite ends from one another (and on opposite sides of the axle 218). Although the trigger 230 is preferably biased into a non-activated state via a spring in the valve assembly 240 or otherwise to, accidental activation of the valve assembly 240 is further prevented via a safety strut 236 that connects to the actuator 232 at its distal end and includes a foot 238 or other interface point at its proximal end. The foot 238 is normally braced against the exterior of the top housing half 212. One or both ends of the safety strut 236 effectively as a living hinge such that when sufficient force is applied to the actuator 232, the safety strut 236 is deformed such that the actuator 232 can be moved toward the top exterior surface of the top housing half 212. The trigger 230 then rotates around the axle 218, causing the valve interface 234 of the trigger to activate the valve assembly 240 such that personal protection spray may be sprayed into the environment.

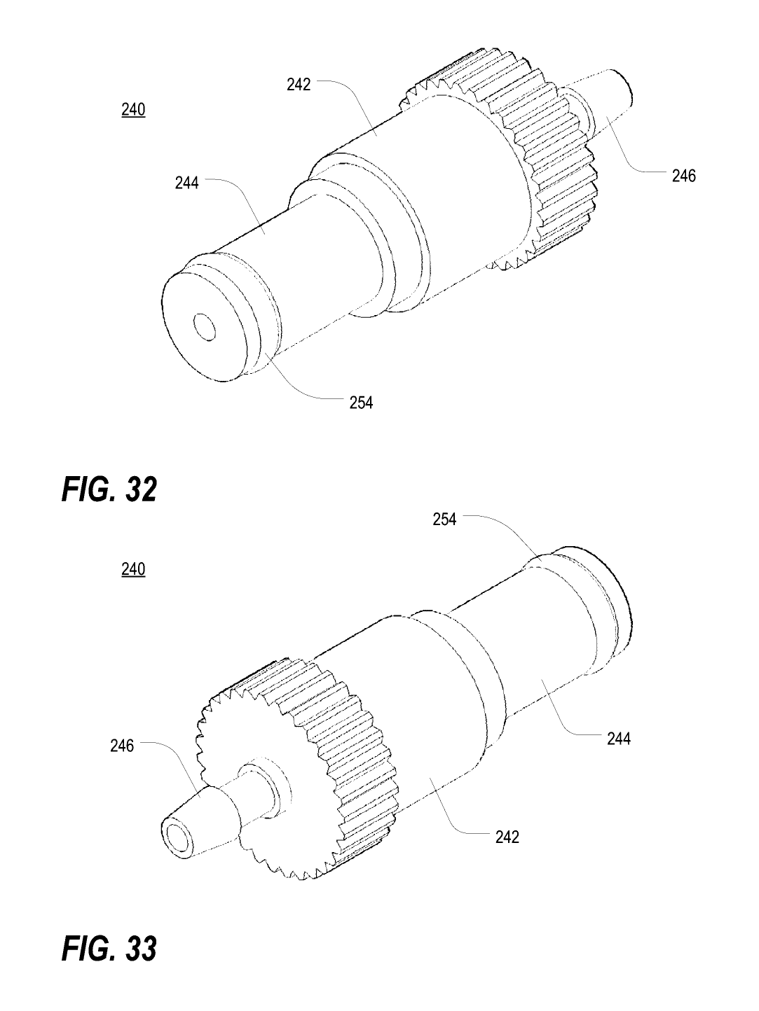

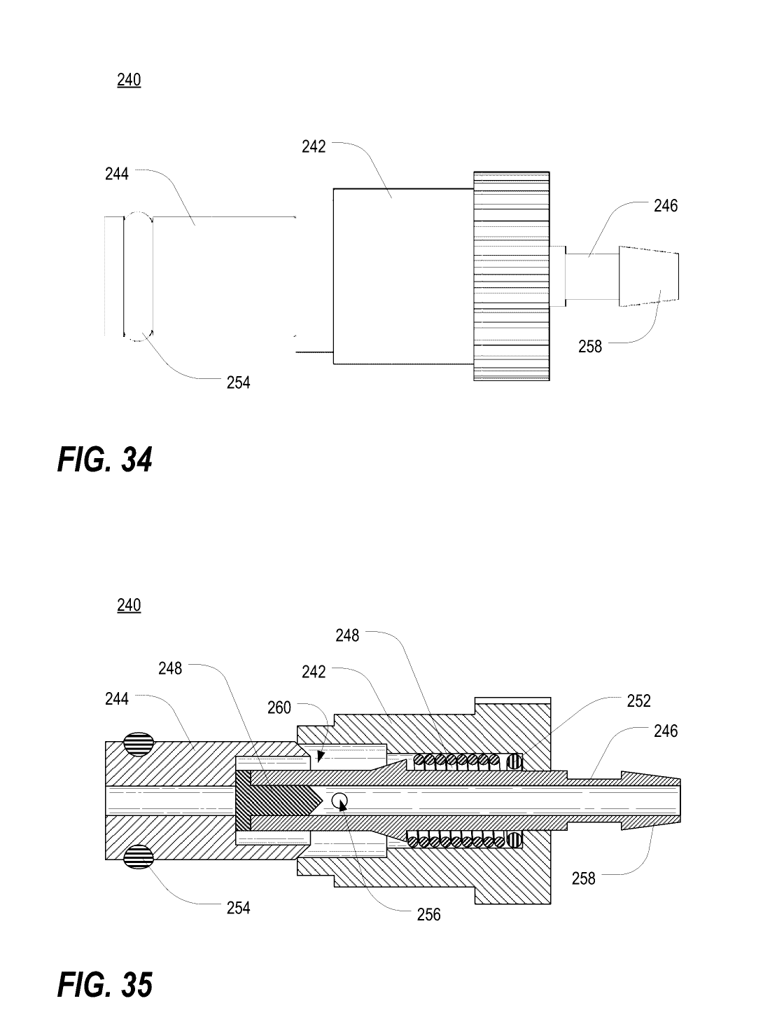

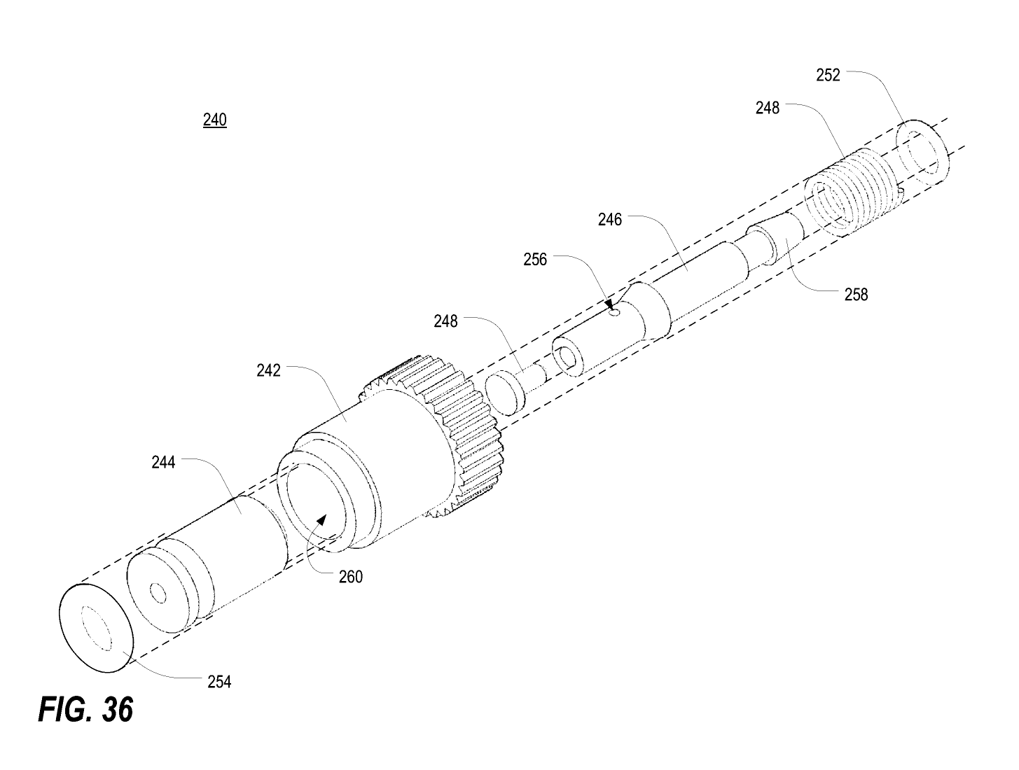

FIGS. 32 and 33 are front and rear isometric views of the valve assembly 240 of FIG. 28, shown in an operational state, while FIGS. 34-36 are a side view, a side cross-sectional view, and an exploded view of the valve assembly 240 of FIGS. 32 and 33. As shown therein, the valve assembly 240 includes a valve body 242, a valve release base 244, a valve release line 246, a bumper 248, a spring 250, and a pair of O-rings 252,254. The valve release line 246 includes a nozzle 258 at one end and a bumper 248 disposed within the opposite end thereof. The valve release line 246 also includes an aperture in a side thereof such that the interior of the valve release line is in fluid communication with an internal chamber 260 of the valve body 242. The spring 250 biases the valve release line 246 relative to the valve body 242. One O-ring 252 prevents leakage from the valve body 242 to the environment while the other O-ring 254 prevents leakage from the interior of the valve assembly 240 back into the interior of the unit housing 210.

The entire valve assembly 240 is arranged within the various cylindrical chambers of the bottom housing half 214 such that portions of the valve body 242 and valve release line 246 extend therefrom at the forward end of the unit 200. As shown in FIG. 28, the nozzle 258 is disposed forward of the valve interface 234 such that a neck of the nozzle fits within the fork 268 of the valve interface 234. Thus, when the trigger 230 is actuated by depressing the actuator 232 with sufficient force to overcome the bias of the spring 248 and the resilient force of the safety strut 236, the nozzle 258 and the rest of the valve release line 246 are pulled forward, away from (out of) the front of the housing 210, and the fluid contained within the valve assembly 240 is released into the environment in the generally forward direction. Subsequent release of the trigger 230 then allows the valve release line 246 to retract within the housing (as biased by the spring 248) and, in at least some embodiments, allows the interior of the valve assembly 240 to refill for another actuation/release operation.

Based on the foregoing information, it will be readily understood by those persons skilled in the art that the present invention is susceptible of broad utility and application. Many embodiments and adaptations of the present invention other than those specifically described herein, as well as many variations, modifications, and equivalent arrangements, will be apparent from or reasonably suggested by the present invention and the foregoing descriptions thereof, without departing from the substance or scope of the present invention.

Accordingly, while the present invention has been described herein in detail in relation to one or more preferred embodiments, it is to be understood that this disclosure is only illustrative and exemplary of the present invention and is made merely for the purpose of providing a full and enabling disclosure of the invention. The foregoing disclosure is not intended to be construed to limit the present invention or otherwise exclude any such other embodiments, adaptations, variations, modifications or equivalent arrangements; the present invention being limited only by the claims appended hereto and the equivalents thereof.

What is claimed is:

1. A wristband-mounted wearable personal protection spray unit, comprising:
(a) a wristband;
(b) a slide cartridge carried on the wristband;
(c) a vessel, containing personal protection spray, that fits inside the slide cartridge; and
(d) a mechanism, integrated with the wristband, for activating the vessel to release the personal protection spray contained therein.

2. The wristband-mounted wearable personal protection spray unit of claim 1, wherein the mechanism includes a vessel tab and a cartridge tab that are activated by squeezing them together.

3. The wristband-mounted wearable personal protection spray unit of claim 1, wherein the personal protection spray includes capsaicin.

4. The wristband-mounted wearable personal protection spray unit of claim 1, wherein the personal protection spray includes phenacyl chloride.

5. The wristband-mounted wearable personal protection spray unit of claim 1, wherein the personal protection spray is an aerosol self-defense spray.

6. A fluid dispenser apparatus comprising:
a sealed fluid reservoir container storing a volume of a fluid under a pressure higher than at least 140 psi;
a nozzle juxtaposed with the sealed fluid reservoir container, and having a fluid inlet that is configured to fluidly couple with the volume of the sealed fluid reservoir container and a fluid outlet configured to emit a stream of the fluid; and
a trigger mechanically coupled with the nozzle and configured, upon activation, to cause the fluid inlet of the nozzle to permanently unseal the sealed fluid reservoir container and relate substantially all the fluid under pressure in the volume through the fluid outlet as the stream in a direction determined by a user.

7. The apparatus of claim 6, wherein the fluid reservoir container comprises a non-cylindrical geometry defining the volume.

8. The apparatus of claim 6, wherein the fluid reservoir container comprises a substantially ovoid geometry defining the volume.

9. The apparatus of claim 8, wherein the ovoid comprises a maximum dimension of no more than 50 mm.

10. The apparatus of claim 6, wherein the fluid reservoir container comprises at least one depression sized and dimensioned to receive at least a portion of the nozzle.

11. The apparatus of claim 6, wherein the volume is no more than about 15 ml.

12. The apparatus of claim 11, wherein the volume is no more than about 4 ml.

13. The apparatus of claim 12, wherein the volume is no more than about 4 ml.

14. The apparatus of claim 6, further comprising a housing that contains at least the reservoir.

15. The apparatus of claim 14, wherein the housing comprises a cover and a detachable base, where the cover releasably couples to the detachable base and permits replacing of the fluid reservoir container.

16. The apparatus of claim 14, further comprising a clip holder coupled to the housing and a clip configured to couple with the clip holder.

17. The apparatus of claim 14, further comprising an appendage strap configured to couple to the housing and sized and dimensioned to strap the housing to a human wrist.

18. The apparatus of claim 1, wherein the fluid inlet comprises a needle portion configured to pierce the sealed fluid upon activation by the trigger.

19. The apparatus of claim 18, wherein the fluid inlet is shaped to depress an internal valve releasing the pressurized contents of reservoir upon activation by the trigger.

20. The apparatus of claim 1, wherein the irritant comprises a pepper derived irritant and a marking component.
